(19) United States
(12) Patent Application Publication
(10) Pub. No.: US 2020/0345251 A1
(43) Pub. Date: Nov. 5, 2020

(54) METHOD AND SYSTEM FOR DETERMINING HEART RATE VARIABILITY

(71) Applicants: Tiago Henrique FALK, Gatineau (CA); Diana Patricia Tobon VALLEJO, Pinares, Cartago-Valle del Cauca (CO); Srinivasan JAYARAMAN, Montréal (CA)

(72) Inventors: Tiago Henrique FALK, Gatineau (CA); Diana Patricia Tobon VALLEJO, Pinares, Cartago-Valle del Cauca (CO); Srinivasan JAYARAMAN, Montréal (CA)

(21) Appl. No.: 16/472,464

(22) PCT Filed: Dec. 22, 2017
(86) PCT No.: PCT/IB2017/058350

§ 371 Date: Jun. 21, 2019

Related U.S. Application Data
(60) Provisional application No. 62/444,534, filed on Jan. 10, 2017.

(51) Int. Cl. A61B 5/024; A61B 5/0245; A61B 5/04; A61B 5/00; G16H 40/63; G16H 50/30; G06F 17/14

(52) U.S. Cl. CPC A61B 5/02405; A61B 5/0245; A61B 5/04014; G06F 17/142; G16H 40/63; G16H 50/30; A61B 5/7257

(57) ABSTRACT

There is described a method for evaluating a heart rate variability comprising: receiving an electrocardiogram representing an electrical activity of a heart; determining a modulation spectrum for the electrocardiogram, the modulation spectrum comprising a plurality of per-frame modulation spectrograms each representing a frequency as a function of a modulation frequency; for each per-frame modulation spectrogram, determining a respective central modulation frequency; determining an indication of the heart rate variability using the determined respective central modulation frequencies for the per-frame modulation spectrograms, a mean central modulation frequency and the number of per-frame modulation spectrograms; and outputting the indication of the heart rate variability.

### TECHNICAL FIELD

The present invention relates to the field of methods and systems for analyzing electrocardiograms, and more particularly to methods and systems for determining heart rate variability.

### BACKGROUND

According to the American Heart Association, cardiovascular disease is one of the major causes of death globally with 17.3 million deaths per year. The number of deaths is expected to grow to more than 23.6 million by 2030. As such, electrocardiogram (ECG) monitoring technologies are burgeoning and portable/wearable devices have emerged, as well as numerous diagnostic and quantified self-applications. Typically, heart rate variability (HRV) analysis has been used as a viable technique for non-invasive assessment of the automatic nervous system (ANS), both in healthy individuals and in patients with cardiac disorders. The ANS is comprised of the sympathetic and parasympathetic nervous systems, where the former prepares the body for action and maintains homeostasis, whilst the latter stimulates the body for relaxation. In response to such ANS activity, heartbeat intervals fluctuate causing a change in the variability of the heart rhythm. This variability has been shown to be related to cardiac autonomic function regulation, as well as to stress, anxiety, diabetes, hypertension, fatigue, and drowsiness, to name a few factors.

Over the years, several indices have been proposed to measure HRV, including linear (time domain, spectral domain) and non-linear methods (e.g., geometric method, entropy, and fractal dynamic methods). Time domain methods typically evaluate the variability in ECG peak-to-peak intervals (also known as ‘RR’ periods) and in the so-called normal-to-normal (NN) intervals between adjacent QRS complexes resulting from sinus node depolarizations. As such, indices such as the standard deviation of the heart rate (i.e., inverse of the RR periods, termed sdHR), standard deviation of all NN intervals (SDNN), and standard deviation of the averages of NN intervals in all 5 min segments of the entire recording (SDANN) have been used. Frequency domain methods, in turn, apply either auto-regressive models or Welch periodogram analysis on the RR interval series in different frequency bands, such as ultra-low frequency (ULF, ≤0.003 Hz), very low frequency (VLF, 0.003-0.04 Hz), low frequency (LF, 0.04-0.15 Hz), and high frequency (HF, 0.15-0.40 Hz). Lastly, nonlinear methods have been proposed and shown to better characterize the complex dynamics of the cardiac autonomic system. As such, geometric methods have been used to convert the RR intervals into a geometric form (e.g. triangle or ellipse), thus representing different classes of HRV. Other non-linear measures include indices of the signal “randomness” (entropy), with measures such as approximate, sample, multiscale, fuzzy, and fuzzy measure entropy, as well as statistical properties of fractals.

Despite the method used to measure HRV, one commonality between all methods is their sensibility to ECG artifacts, particularly movement artifacts. Such limitations have been reported for time and frequency domain measures, as well as non-linear ones. Erroneous HRV measurements resultant from noisy ECG signals can have severe detrimental effects, including a rise in false alarms in automated health monitoring devices. A common practical approach taken to alleviate this shortcoming is to perform ECG enhancement (i.e., artifact removal) prior to HRV index calculation. Typically, wavelet-based enhancement algorithms have been used for this purpose. However, alternate strategies are still needed in very noisy scenarios, as ECG enhancement algorithm performance is limited in such settings.

Therefore, there is a need for an improved method and system for determining heart rate variability.

### SUMMARY

According to a first broad aspect, there is provided a computer-implemented method for evaluating a heart rate variability comprising: receiving an electrocardiogram representing an electrical activity of a heart; determining a modulation spectrum for the electrocardiogram, the modulation spectrum comprising a plurality of per-frame modulation spectrograms each representing a frequency as a function of a modulation frequency; for each per-frame modulation spectrogram, determining a respective central modulation frequency; determining an indication of the heart rate variability using the determined respective central modulation frequencies for the per-frame modulation spectrograms, a mean central modulation frequency and the number of per-frame modulation spectrograms; and outputting the indication of the heart rate variability.

In one embodiment, said determining the modulation spectrum comprises: applying a first transform to the electrocardiogram, thereby obtaining a time frequency representation of the electrocardiogram; and applying a second transform across a time dimension of the time frequency representation, thereby obtaining the modulation spectrum.

In one embodiment, the method further comprises dividing the electrocardiogram into a plurality of overlapping time windows before applying the first transform and dividing the time frequency representation into a plurality of overlapping modulation windows before applying the second transform.

In one embodiment, the first transform is a first fast Fourier transform.

In one embodiment, the second transform is a second fast Fourier transform.

In one embodiment, said determining the respective central modulation frequency comprises determining a central modulation frequency of a first lobe contained in the per-frame modulation spectrogram.

In one embodiment, the method further comprises identifying the first lobe for each per-frame modulation spectrogram.

In one embodiment, the method further comprises calculating the mean central modulation frequency.

In one embodiment, said determining the indication of the heart rate variability comprises calculating a standard deviation of the central modulation frequencies of the per-frame modulation spectrograms.

In one embodiment, the method further comprises measuring the electrocardiogram.

According to another broad aspect, there is provided a system for evaluating a heart rate variability, comprising: a spectrum calculator for receiving an electrocardiogram representing an electrical activity of a heart and determining a modulation spectrum for the electrocardiogram, the modulation spectrum comprising a plurality of per-frame modulation spectrograms each representing a frequency as a function of a modulation frequency; a central frequency calculating unit for determining, for each per-frame modulation spectrogram, a respective central modulation frequency; and a heart rate variability determining unit for determining an indication of the heart rate variability using the determined respective central modulation frequencies for the per-frame modulation spectrograms, a mean central modulation frequency and the number of per-frame modulation spectrograms and outputting the indication of the heart rate variability.

In one embodiment, the spectrum calculator is adapted to apply a first transform to the electrocardiogram, thereby obtaining a time frequency representation of the electrocardiogram and apply a second transform across a time dimension of the time frequency representation, thereby obtaining the modulation spectrum.

In one embodiment, the spectrum calculator is further adapted to divide the electrocardiogram into a plurality of overlapping time windows before applying the first transform and divide the time frequency representation into a plurality of overlapping modulation windows before applying the second transform.

In one embodiment, the central frequency calculating unit is adapted to determine a central modulation frequency of a first lobe contained in the per-frame modulation spectrogram.

In one embodiment, the central frequency calculating unit is further adapted to identify the first lobe for each per-frame modulation spectrogram.

In one embodiment, the heart rate variability determining unit is further adapted to calculate the mean central modulation frequency.

In one embodiment, the heart rate variability determining unit is adapted to calculate a standard deviation of the central modulation frequencies of the per-frame modulation spectrograms.

In one embodiment, the system further comprises a sensor for measuring the electrocardiogram.

### BRIEF DESCRIPTION OF THE DRAWINGS

Further features and advantages of the present invention will become apparent from the following detailed description, taken in combination with the appended drawings, in which:

FIG. 1 is a flow chart illustrating a method for evaluating a heart rate variability, in accordance with an embodiment;

FIG. 2 is a block diagram illustrating a system for evaluating a heart rate variability, in accordance with an embodiment;

FIG. 3 is a block diagram of a processing module adapted to execute at least some of the steps of the method of FIG. 1, in accordance with an embodiment.

FIG. 4. illustrates exemplary signal processing steps involved in the spectro-temporal ECG representation in which an ECG signal, x(n), is first segmented and transformed into the time-frequency representation via 512-point FFT, modulation spectral magnitudes are then segmented and transformed via a second transform (512-point FFT) into a frequency-frequency representation and the right part of FIG. 3 shows modulation spectrograms frames from 1 to k;

FIG. 5 illustrates exemplary modulation spectrograms of synthesized ECG with (a) clean 120 beats per minute (bpm) and its noisy counterparts signals with an (b) signal to noise ratio (SNR)=0 dB, and (c) SNR=−10 dB;

FIG. 6 illustrates exemplary five-second excerpts from a synthetic ECG signals with 120 bpm for clean and two noisy counterparts with an SNRs of 0 dB and −10 dB;

FIG. 7 illustrates exemplary scatterplots for noisy synthetic ECG signals with 100 bpm and SNR=−10 dB between (a) ‘true’ sdHR and noisy sdHR, (b) ‘true’ sdHR and wavelet enhanced sdHR, and (c) ‘true’ sdHR and proposed MD-HRV;

FIG. 8 illustrates exemplary Bland-Altman plots for noisy synthetic ECG signals with 100 bpm and SNR=−10 dB between (a) ‘true’ sdHR and noisy sdHR, (b) ‘true’ sdHR and wavelet enhanced sdHR, and (c) ‘true’ sdHR and proposed MD-HRV;

FIG. 9 illustrates exemplary scatterplots for recorded ECG signals for (a) Noisy sdHR and ‘true’ sdHR, (b) Wavelet enhanced sdHR and ‘true’ sdHR, and (c) Proposed MD-HRV and ‘true’ sdHR; and

FIG. 10 illustrates exemplary Bland-Altman plots for recorded ECG signals for (a) Noisy sdHR and ‘true’ sdHR, (b) Wavelet enhanced sdHR and ‘true’ sdHR, and (c) Proposed MD-HRV and ‘true’ sdHR.

It will be noted that throughout the appended drawings, like features are identified by like reference numerals.

### DETAILED DESCRIPTION

In the following, there is described a method of measuring HRV that is robust to ECG artifacts, even in some very noisy cases. The method uses a spectro-temporal signal processing technique termed “modulation spectrum processing,” which measures the rate-of-change of ECG short-term spectral magnitude components. The method allows the determination of a metric, termed modulation-domain HRV (MD-HRV), which estimates the heart rate (and consequently HRV) in extremely noisy cases, without the need for a priori ECG enhancement.

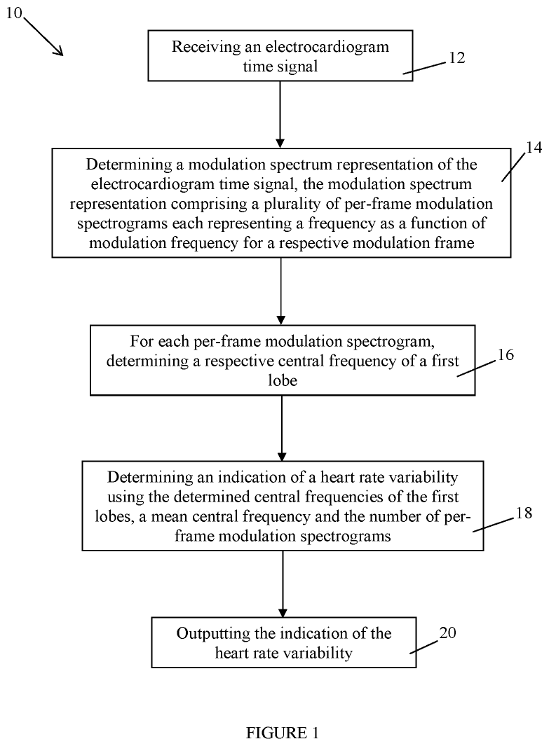

FIG. 1 illustrates one embodiment of a computer-implemented method 10 for determining a heart rate variability. The method 10 is performed by at least one processor or processing unit that is connected to a memory and a communication unit for receiving and transmitting data. The first step 12 consists in receiving a measured time-domain ECG signal, i.e. a time-domain signal of which the amplitude is representative of the amplitude of the electrical activity of a heart measured by an adequate sensor, via the communication unit. The ECG signal may be received from a computing machine such as personal computer, a server, a smartphone, or a tablet via a telecommunication network or a wired or wireless connection. In this case, the ECG signal is measured by the adequate sensor and then transmitted and stored on the computing machine. The ECG signal may also be directly received from the sensor that may be embedded in devices such as a smartwatch, an ECG chest strap, an ECG recording unit or the like. The received time-domain ECG signal may be an electrical signal such as a digital signal or an analog signal.

At step 14, a modulation spectrum representation of the received ECG signal is determined by the processor. The modulation spectrum of a time-domain signal presents the frequency of the signal as a function of the modulation frequency which represents the rate of change of a signal frequency. Therefore, the modulation spectrum provides the modulation frequency, i.e. the rate of change, for each frequency contained in the ECG signal. The modulation spectrum representation comprises a plurality of per-frame modulation spectrograms which each represents the frequency as a function of the modulation frequency for a respective frame and each per-frame modulation spectrogram comprises a plurality of lobes.

In one embodiment, two transforms, i.e. a first or base transform and a second or modulation transform, are successively applied to the time-domain ECG signal to obtain its modulation spectrum. In one embodiment, two Fourier transforms are successively applied to the time-domain ECG signal in order to obtain its modulation spectrum. A first Fourier transform is applied to the time-domain ECG signal in order to obtain a time-frequency representation. Then, a second and different Fourier transform is applied across the time dimension of the time-frequency representation to obtain the modulation spectrum, which is a frequency-frequency modulation representation.

While the present description refers to the use of first and second Fourier transforms to convert the time-domain ECG into the time-frequency domain and the time-frequency domain signal into a frequency-frequency modulation signal, respectively, it should be understood that first and second transforms other than Fourier transforms may be used. Any adequate first transform that may convert the time-domain ECG into a frequency-domain signal may be used in replacement of the first Fourier transform. Similarly, any adequate second transform that may convert a frequency-domain signal into a frequency-frequency modulation signal may be used in replacement of the second Fourier transform. For example, the first transform may be a Fourier transform while the second transform may be a lapped transform or a wavelet transform. In one embodiment, the first and/or second transform(s) may be invertible.

In one embodiment, the ECG signal is divided into a plurality of overlapping time windows or segments with an overlapping ratio OR₁ and the first or base transform is applied to the divided ECG signal to obtain a frequency domain signal which represents the frequency of the ECG signal as a function of time. Similarly, the frequency domain signal is divided into a plurality of overlapping modulation windows or segments with an overlapping ratio OR₂ and the second or modulation transform is applied to the divided frequency domain signal to obtain the frequency-frequency modulation signal. In one embodiment, the overlapping ratios OR₁ and OR₂ are chosen as follows:
OR₁ϵ[0, 100 [%
OR₂ϵ[0, 100 [%

Once the modulation spectrum of the ECG signal has been determined, the next step 16 consists in determining the central modulation frequency of the first lobe contained in each per-frame modulation spectrogram for each per-frame modulation spectrogram. The central modulation frequency of the first lobe, multiplied by 60, corresponds to the number of beats per second (i.e., a central modulation frequency of 1 Hz corresponds to an instantaneous heart rate of 60 beats per minute).

It should be understood that any adequate method for determining the central modulation frequency of the first lobe of a per-frame modulation spectrogram may be used. For example, the different lobes contained in a per-frame modulation spectrogram may first be determined before isolating the first lobe. The central modulation frequency of the first lobe may be determined by identifying the maximum frequency amplitude of the lobe, the central modulation frequency corresponding to the modulation frequency of the maximum frequency amplitude. In another example, the minimum and maximum modulation frequencies of the first lobe may be determined and the central modulation frequency of the first lobe may be determined from the minimum and maximum modulation frequencies of the first lobe.

Referring back to FIG. 1, an indication of the HRV is determined at step 18. The mean central modulation frequency of the first lobe for the different per-frame modulation spectrograms is determined using the previously determined central frequency of the first lobe of each per-frame modulation spectrogram. The indication of the HRV is then determined based on the number of per-frame modulation spectrograms, the mean central modulation frequency and the central modulation frequency of the first lobe of each per-frame modulation spectrogram using Equation 1 provided below. In one embodiment, the indication of the HRV corresponds to the standard deviation of the central modulation frequencies of the first lobes of the per-frame modulation spectrograms.

At step 20, the indication of the HRV is outputted. For example, the indication of the HRV may be stored in memory such as a cache memory, or outputted by the communication unit to be displayed on a display unit.

In one embodiment, the method 10 further comprises the step of measuring the ECG using any adequate sensor.

It should be understood that the above-described method may be embodied as a system comprising at least one processor or processing unit, a memory or storing unit, and a communication unit for receiving and transmitting data. The memory comprises statements and instructions stored thereon that, when executed by the processor, performs the steps of the above-described method 10.

In one embodiment, the method 10 is embodied as a computer program product which comprises a computer readable memory storing computer executable instructions and statements thereon that, when executed by a processing unit, perform the steps of the method 10.

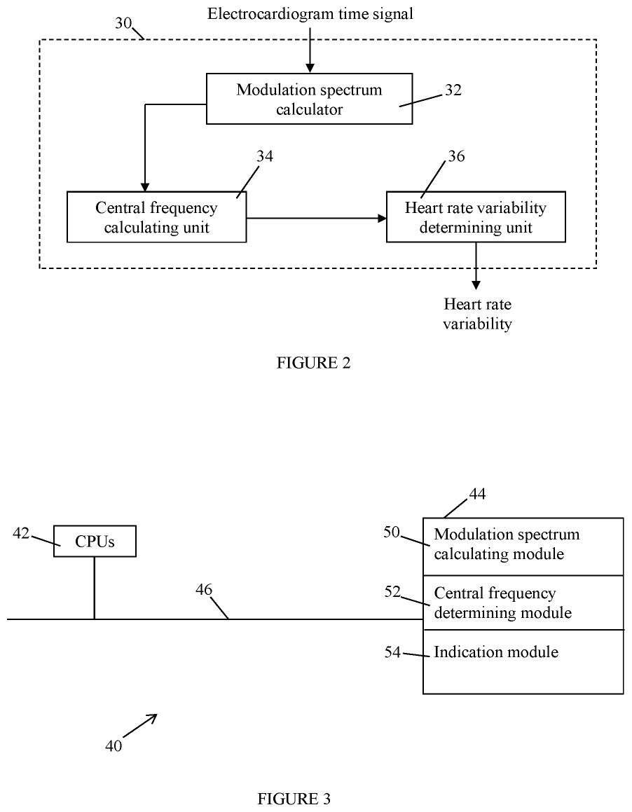

FIG. 2 illustrates one embodiment of a system 30 for evaluating the HRV of a measured ECG. The system 30 comprises a modulation spectrum calculator 32, a central frequency calculating unit 34, and a heart rate variability or HRV determining unit 36.

The modulation spectrum calculator 32 is adapted to receive a measured ECG signal that corresponds to a time representation of a heart rate measured by an adequate sensor and determine a modulation spectrum for the time domain ECG signal using the above-described method.

The central frequency calculating unit 34 is adapted to receive the modulation spectrum from the modulation spectrum calculator 32 and determine the central modulation frequency of the first lobe for each per-frame modulation spectrogram constituting the modulation spectrum using the above-described method.

The HRV determining unit 36 is adapted to receive the central modulation frequency of the first lobe for each per-frame modulation spectrogram from the central frequency calculating unit 34 and the number of per-frame modulation spectrograms from the modulation spectrum calculator 32. The HRV determining unit 36 is further adapted to determine the mean central modulation frequencies of the first lobes and calculate an indication of the HRV using the above-described method.

In one embodiment, the modulation spectrum calculator 32, the central frequency calculating unit 34, and the HRV determining unit 36 are independent from one another, and they are each provided with respective processing unit, internal memory, and communication unit. The internal memory of each one of the modulation spectrum calculator 32, the central frequency calculating unit 34, and the HRV determining unit 36 comprises statements and instructions stored thereon that, when executed by the respective processing unit, performs the respective steps of the method 10. In another embodiment, the modulation spectrum calculator 32, the central frequency calculating unit 34, and the HRV determining unit 36 are all part of a same device which comprises a processing unit, a memory, and communication means. In this case, the processing unit is adapted to execute all of the steps of the method 10.

It should also be understood that the ECG signal may be generated by any adequate measuring device adapted to measure an ECG. In another example, the ECG signal may be a synthetized signal. In this case, the ECG signal does not correspond to a signal measured on a body.

In one embodiment, each one of the modulation spectrum calculator 32, the central frequency calculating unit 34 and the heart rate variability determining unit 36 is provided with a respective processing unit, a respective memory and respective communication means. In another embodiment, the modulation spectrum calculator 32, the central frequency calculating unit 34 and/or the heart rate variability determining unit 36 may share a same processing unit, a dame memory and/or same communication means. It should be understood that a processing unit may comprise at least one processor such as a microprocessor, an integrated circuit, or the like.

It should be understood that at least some of the steps of the method 10 may be performed by a computer machine provided with at least one processing unit, a memory or storing unit, and communication means. The memory comprises statements and instructions stored thereon that, when executed by the processing unit, performs at least some of the steps of the method 10.

FIG. 3 is a block diagram illustrating an exemplary processing module 40 for executing the steps 12 to 20 of the method 10, in accordance with some embodiments. The processing module 40 typically includes one or more Computer Processing Units (CPUs) and/or Graphic Processing Units (GPUs) 42 for executing modules or programs and/or instructions stored in memory 44 and thereby performing processing operations, memory 44, and one or more communication buses 46 for interconnecting these components. The communication buses 46 optionally include circuitry (sometimes called a chipset) that interconnects and controls communications between system components. The memory 44 includes high-speed random access memory, such as DRAM, SRAM, DDR RAM or other random access solid state memory devices, and may include non-volatile memory, such as one or more magnetic disk storage devices, optical disk storage devices, flash memory devices, or other non-volatile solid state storage devices. The memory 44 optionally includes one or more storage devices remotely located from the CPU(s) 42. The memory 44, or alternately the non-volatile memory device(s) within the memory 44, comprises a non-transitory computer readable storage medium. In some embodiments, the memory 44, or the computer readable storage medium of the memory 44 stores the following programs, modules, and data structures, or a subset thereof:
an modulation spectrum calculating module 50 for receiving an electrocardiogram and determining a modulation spectrum representation;
a central frequency determining module 52 for determining a central frequency of a first lobe; and
an indication module 54 for determining an indication of a heart rate variability.

Each of the above identified elements may be stored in one or more of the previously mentioned memory devices, and corresponds to a set of instructions for performing a function described above. The above identified modules or programs (i.e., sets of instructions) need not be implemented as separate software programs, procedures or modules, and thus various subsets of these modules may be combined or otherwise re-arranged in various embodiments. In some embodiments, the memory 44 may store a subset of the modules and data structures identified above. Furthermore, the memory 44 may store additional modules and data structures not described above.

Although it shows a processing module 40, FIG. 3 is intended more as functional description of the various features which may be present in a management module than as a structural schematic of the embodiments described herein. In practice, and as recognized by those of ordinary skill in the art, items shown separately could be combined and some items could be separated.

In the following, there is described an exemplary application of the method 10 for determining the HRV of an ECG signal. The exemplary method allows for the determination of a new noise-robust HRV index termed MD-HRV (modulation-domain HRV), based on a spectro-temporal ECG signal representation. More specifically, by quantifying the rate-of-change of ECG spectral components over time, heart rate estimates can be reliably obtained even in extremely noisy signals, thus bypassing the need for ECG enhancement. Experiments with synthetic ECG signals corrupted at various different signal-to-noise levels, as well as recorded noisy signals show the proposed measure outperforming several HRV benchmark parameters computed post wavelet-based enhancement. These findings suggest that the proposed MD-HRV metric is well-suited for ambulant cardiac monitoring applications, particularly those involving intense movement.

The remainder of the present description is organized as follows. Section I provides a description of the ECG modulation spectral representation, the proposed MD-HRV metric, benchmark HRV metrics, and performance figures-of-merit. Experimental results are then presented in Section II and discussed in Section III. Finally, conclusions are drawn in Section IV.

### I. Methods and Materials

In this section, there is presented the spectro-temporal ECG representation and the proposed MD-HRV metric extraction approach is described. Furthermore, a description of the synthetic and realistic datasets used, HRV metrics in time and frequency domains, as well as nonlinear measures are introduced. Finally, figures-of-merit to gauge MD-HRV performance are presented.

### A. Spectro-Temporal ECG Representation

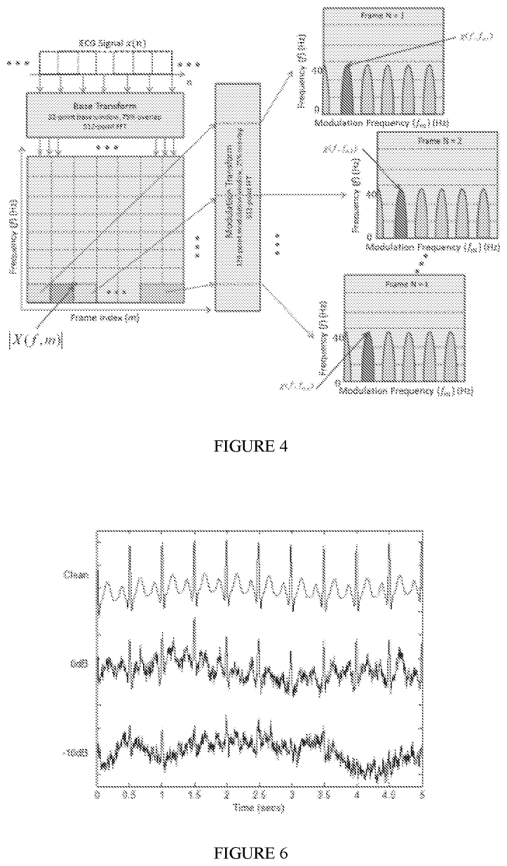

The processing steps for the computation of the spectro-temporal ECG representation are depicted in FIG. 4. First, the ECG signal x(n), sampled at 256 Hz, is segmented into overlapping frames employing a 32-point sine window and 75% overlap to be then transformed to the frequency domain (spectrogram) via a 512-point fast Fourier transform (FFT). Then, the magnitude of the spectral components |X(f, m)| (f being the conventional frequency in Hertz (Hz) and m being the frame index) is segmented across time using a 128-point sine window with 75% overlap to obtain the frequency-frequency representation X(f, f_{m,k}) via a 512-point FFT. While alternate parameter configurations (e.g., base window, modulation window, overlap, and number of FFT points) can be used, these particular values were empirically found to be optimal for a sampling rate of 256 Hz. The frequency-frequency representation is known as modulation spectrogram as described above, where f_{m,k} is the modulation frequency in Hertz (Hz) and k is the frame index for the second FFT. Synthesized clean ECG data were used to optimize the base and modulation window sizes, as well as the overlap rates reported above.

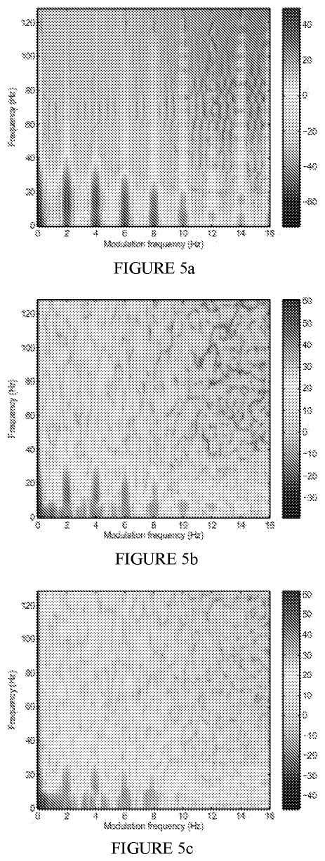

The modulation spectrogram shows the rate-of-change of the different ECG spectral components. FIG. 5 (a) shows a typical modulation spectrogram over 5 seconds of a synthetic clean ECG signal with 120 beats-per-minute (bpm). FIGS. 5 (b) and 5(c), in turn, depict its noisy counterparts with a signal-to-noise ratio (SNR) of 0 dB and −10 dB, respectively. As can be seen from FIG. 5 (a), the first “lobe” centred at 2 Hz corresponds to the ECG heart rate, followed by several harmonics across the modulation frequency axis. While the first lobe energy decreases as the signal becomes noisier, it is still possible to detect it even at SNR=−10 dB (FIG. 5 (c)). The corresponding time domain waveforms can be seen in FIG. 6. From the bottom plot, it can be seen that time-domain HRV measurement at an SNR=−10 dB could be impossible. The clear advantage of the modulation spectrum for heart rate detection in noisy ECGs is the main motivation for using this representation for HRV measurement, as described next.

### B. Modulation Domain HRV Measurement

As outlined above, the spectro-temporal ECG representation allows distinguishing modulation spectral components even in extremely noisy scenarios, as depicted in FIG. 5 (c). Here, a new modulation-domain HRV metric (MD-HRV) is presented. In order to compute the metric, the central frequency (i.e., the central frequency of the first lobe) within each per-frame modulation spectrogram X*(f, f_{m,k}) has to be detected; this is represented by the shaded lobes in the modulation spectrograms of FIG. 4. For this purpose, the energy for each modulation frequency bin is calculated between the 0≤f≤40 region where most of the ECG energy is concentrated. The modulation frequency bin with the highest energy between 0.8≤fₘ≤3.3 is selected as the center of the first lobe. The fₘ range was chosen to cover heart rates between 48 and 198 bpm, thus encompassing different cardiac diseases and activity levels. Notwithstanding, this parameter can be easily updated to incorporate HR values outside this range. Next, the standard deviation of the per-frame central frequencies (i.e., “instantaneous” HRs) is calculated and used as a metric of heart rate variability. For the sake of notation, let X*ₖ=X*(f, f_{m,k}), thus the MD-HRV over all per-frame modulation spectrograms and given in bpm is computed as:

$$MD - HRV ( bpm ) = 60 \times \sqrt{\frac{1}{N - 1} {\sum }_{k = 1}^{N} {( {𝒳}_{k}^{*} - {\overline{𝒳}}^{*} )}^{2}} , \qquad ( 1 )$$

where N is the total number of per-frame modulation spectrograms or ‘modulation frames’, X*ₖ is the central modulation frequency (ie, HR) at the k^{th} modulation frame, and X̅* is the mean central modulation frequency calculated over all per-frame modulation spectrograms. The multiplying factor of 60 is used to convert the MD-HRV metric from Hz to bpm. Here, modulation spectrograms are estimated every 5 seconds with 75% overlap. While alternate parameter configurations (e.g., base window, modulation window, overlap, and number of FFT points) can be used, these particular values were empirically found to be optimal for a sampling rate of 256 Hz. For this particular configuration, a 5-second duration ECG signal will generate an RR series with five points.

### C. Dataset 1: Synthetic ECG

Synthetic ECG signals were generated using the ‘ecgsyn’ function in MATLAB™ available in PHYSIONET™. The function uses a dynamic model to generate the ECG waveform and allows configuring several parameters such as mean heart rate, sampling frequency, standard deviation of the heart rate, waveform morphology (i.e., P, Q, R, S, and T duration, timing, and amplitude), and low-frequency (LF) to high frequency (HF) ratio. Here, in total 700 signals of 10-minute duration sampled at 256 Hz were generated by randomly sampling the heart rate standard deviation between 1 and 10 bpm for heart rates ranging from 50 to 180 bpm.

The heart rate standard deviation and heart rate ranges were chosen in order to cover cardiac diseases such as bradycardia, tachycardia, and arrhythmia as well as different activity levels (i.e., resting, walking, and running). Clean synthetic signals were corrupted by several artifacts at known SNR levels.

Baseline wander noise, and muscle artifacts were taken from MIT-BIH Noise Stress Test Dataset. Moreover, pink noise was added to model observation noise, as well as Brownian noise to model electrode movement artifacts. Noisy signals were generated at 6 different SNR levels of −10 dB, −8 dB, −5 dB, 0 dB, 5 dB, and 10 dB. FIG. 6 depicts synthesized 5-second excerpts of a clean (top) and two noisy signals at SNR=−10 dB (bottom) and 0 dB (middle). Overall, 4200 noisy 10-minute signals (700 hours) were generated for HRV metric assessment.

### D. Dataset 2: Recorded MIT-BIH Arrhythmia Dataset

In order to test the proposed HRV metric in realistic pathological signals, the recorded MIT-BIH Arrhythmia database was also used. Data sampled at 360 Hz were taken from 48 patients by the BIH Arrhythmia Laboratory, comprising two-channel ambulatory ECG recordings. Two cardiologists analyzed independently the data, where disagreements were resolved to obtain annotations for each beat. Each recording has 30-minute length. For the experiments described herein, the two-channel signals were segmented to create 10-minute segments downsampled at 256 Hz. Thus, a total of 288 10-minute segments were created providing an overall of 48 hours for testing. These segments were then grouped according to the heart rate (HR) and heart rate standard deviation (sdHR), similar to the synthetic ECG signals, thus allowing for easier comparison between the two databases. Hence, four groups were found: Group 1 (60±10 bpm), Group 2 (80±10 bpm), Group 3 (100±10 bpm), and Group 4 (120±10 bpm).

### E. Benchmarks and Figures-of-Merit

In order to compare the proposed MD-HRV metric with “gold standard” benchmarks, several time domain, frequency domain, and non-linear HRV metrics were computed from the clean ECG signals. Time-domain HRV metrics include i) the widely-used SDNN, corresponding to the standard deviation of the normal-to-normal (NN) intervals, i.e., reflects cyclic components which cause variability in the ECG signal; ii) SDANN, i.e., the standard deviation of 5-minute mean NN intervals, and iii) standard deviation of heart rate estimates (stdHR) computed after peak detection and given in beats-per-minute (bpm). The frequency-domain metric, in turn, measures the total spectral power contained in frequency sub-bands such as very low frequency (VLF, 0-0.04 Hz), low frequency (LF, 0.04-0.15 Hz), and high frequency (HF, 0.15-0.4 Hz). Lastly, non-linear Poincaré plots are used to quantify self-similarity assuming that each inter-beat interval (IBI) is influenced by the previous one. Thus, an ellipse is fitted with a long axis representing the line of identity as well as a perpendicular axis to it. If the IBIs are longer than the previous ones, the points will be located above the line of identity and below for the opposite case. Consequently, the so-called SD2 parameter represents the standard deviation along the line of identity and the SD1 parameter represents the standard deviation over the perpendicular line. Hence, SD1 indicates the standard deviation of instantaneous beat-to-beat (i.e., short term variability) and SD2 of continuous or long-term variability.

Moreover, a wavelet-based ECG enhancement algorithm was used for artifact removal prior to benchmark HRV measurement. Such setup exemplifies the process used in existing state-of-the-art HRV monitoring applications. Here, comparisons will be made between the proposed MD-HRV metric computed from the noisy signal and benchmark HRV metrics computed from the noisy and enhanced signals. Daubechies-6 mother wavelets with 8 levels of decomposition, combined with soft thresholding and universal shrinkage were used; such settings resulted in the best enhancement performance on the two datasets used herein.

As for figures-of-merit, Pearson (ρ) and Spearman (ρ_{S}) correlations were used to gauge the benefits of the proposed MD-HRV metric. Due to the availability of the original clean synthetic signal and the annotation files for the arrhythmia dataset, the proposed metric could be compared with ‘ground truth’ benchmark HRV values. Pearson correlation shows the linear relationship between MD-HRV and the ‘true’ HRV metrics. Spearman correlation, in turn, measures how well MD-HRV ranks against the ‘true’ metrics. Higher correlation values correspond to improved metrics. Additionally, to evaluate the bias and to estimate an agreement interval between metrics, Bland-Altman plots were also used. A Bland-Altman plot depicts the differences (vertical axis) against the averages (horizontal axis), hence, are used to verify the level of agreement between the ‘true’ metrics and the proposed MD-HRV. Typically, Bland-Altman plots with a more compact point distribution around zero (vertical axis) represent metrics in better agreement. Lastly, linear regression analysis is also performed to show the linear fitting with confidence intervals at 95%; smaller confidence intervals represent more reliable metrics.

### III. Results

Tables I and II show the performance comparison for the different HRV metrics calculated from the noisy signals, their enhanced wavelet counterparts, and the proposed MD-HRV metric for datasets 1 (synthetic) and 2 (recorded), respectively. The Pearson (p) and Spearman (ρ_{S}) correlations were obtained between the ‘true’ benchmark HRV metrics for clean signals (i.e., SDNN, SDANN, sdHR, total power, SD1, and SD2) and HRV metrics for noisy and enhanced signals, as well as between the ‘true’ metrics and the proposed MD-HRV. The results reported in the tables correspond to the average over the four groups, as described in Section II-D.

TABLE I
Performance comparison (ρ and ρ_{S}) of HRV metrics for
noisy signals, wavelet enhanced signals and the proposed
MD-HRV metric for dataset 1 (synthetic) at different SNR levels.

| | | | |
|---|---|---|---|
| Input SNR (dB) | Noisy | Enhanced | MD-HRV |

SDNN

| | | | |
|---|---|---|---|
| −10 | 0.221 ± 0.375 | 0.439 ± 0.368 | 0.781 ± 0.055 |
| −8 | 0.505 ± 0.474 | 0.615 ± 0.383 | 0.924 ± 0.069 |
| −5 | 0.896 ± 0.182 | 0.852 ± 0.281 | 0.969 ± 0.045 |
| 0 | 0.912 ± 0.215 | 0.965 ± 0.091 | 0.979 ± 0.030 |
| 5 | 1.000 ± 0.001 | 0.976 ± 0.062 | 0.988 ± 0.009 |
| 10 | 1.000 ± 0.000 | 1.000 ± 0.000 | 0.990 ± 0.048 |

SDANN

| | | | |
|---|---|---|---|
| −10 | 0.271 ± 0.382 | 0.443 ± 0.353 | 0.751 ± 0.074 |
| −8 | 0.541 ± 0.464 | 0.650 ± 0.353 | 0.915 ± 0.071 |
| −5 | 0.920 ± 0.141 | 0.859 ± 0.266 | 0.962 ± 0.049 |
| 0 | 0.944 ± 0.119 | 0.970 ± 0.061 | 0.970 ± 0.040 |
| 5 | 0.994 ± 0.004 | 0.985 ± 0.027 | 0.980 ± 0.019 |
| 10 | 0.996 ± 0.003 | 0.998 ± 0.001 | 0.982 ± 0.016 |

sdHR

| | | | |
|---|---|---|---|
| −10 | 0.225 ± 0.115 | 0.285 ± 0.346 | 0.762 ± 0.079 |
| −8 | 0.537 ± 0.162 | 0.494 ± 0.314 | 0.924 ± 0.066 |
| −5 | 0.883 ± 0.099 | 0.836 ± 0.213 | 0.969 ± 0.045 |
| 0 | 0.989 ± 0.025 | 0.991 ± 0.015 | 0.980 ± 0.029 |
| 5 | 0.999 ± 0.001 | 0.996 ± 0.006 | 0.990 ± 0.010 |
| 10 | 0.999 ± 0.000 | 0.999 ± 0.001 | 0.991 ± 0.007 |

SD1

| | | | |
|---|---|---|---|
| −10 | 0.156 ± 0.298 | 0.407 ± 0.359 | 0.757 ± 0.076 |
| −8 | 0.443 ± 0.270 | 0.562 ± 0.379 | 0.916 ± 0.070 |
| −5 | 0.821 ± 0.290 | 0.794 ± 0.289 | 0.963 ± 0.041 |
| 0 | 0.802 ± 0.467 | 0.906 ± 0.218 | 0.973 ± 0.027 |
| 5 | 0.969 ± 0.061 | 0.887 ± 0.295 | 0.981 ± 0.008 |
| 10 | 0.993 ± 0.003 | 0.999 ± 0.001 | 0.983 ± 0.005 |

SD2

| | | | |
|---|---|---|---|
| −10 | 0.267 ± 0.415 | 0.450 ± 0.367 | 0.759 ± 0.077 |
| −8 | 0.528 ± 0.521 | 0.638 ± 0.381 | 0.921 ± 0.066 |
| −5 | 0.917 ± 0.126 | 0.864 ± 0.278 | 0.963 ± 0.052 |
| 0 | 0.952 ± 0.110 | 0.980 ± 0.050 | 0.974 ± 0.036 |
| 5 | 0.998 ± 0.004 | 0.991 ± 0.024 | 0.982 ± 0.020 |
| 10 | 0.998 ± 0.004 | 1.000 ± 0.000 | 0.984 ± 0.017 |

Total power (AR)

| | | | |
|---|---|---|---|
| −10 | 0.194 ± 0.265 | 0.346 ± 0.322 | 0.711 ± 0.067 |
| −8 | 0.423 ± 0.355 | 0.524 ± 0.408 | 0.871 ± 0.049 |
| −5 | 0.803 ± 0.273 | 0.760 ± 0.317 | 0.908 ± 0.042 |
| 0 | 0.791 ± 0.329 | 0.889 ± 0.259 | 0.918 ± 0.029 |
| 5 | 0.921 ± 0.018 | 0.922 ± 0.188 | 0.926 ± 0.027 |
| 10 | 0.924 ± 0.023 | 0.998 ± 0.002 | 0.928 ± 0.028 |

TABLE II
Performance comparison (ρ and ρₛ) of HRV
metrics for noisy signals, wavelet enhanced signals
and the proposed MD-HRV metric for dataset 2 (recorded).

| | Noisy | | Enhanced | | MD-HRV | |
|---|---|---|---|---|---|---|
| HRV metrics | ρ | ρₛ | ρ | ρₛ | ρ | ρₛ |
| SDNN | 0.632 | 0.837 | 0.617 | 0.844 | 0.893 | 0.830 |
| SDANN | 0.687 | 0.81 [symbol] | 0.641 | 0.80 [symbol] | 0.899 | 0.814 |
| sdHR | 0.608 | 0.759 | 0.616 | 0.788 | 0.9 [symbol] 2 | 0.834 |
| SD1 | 0. [symbol] 75 | 0.828 | 0.632 | 0.820 | 0.908 | 0.734 |
| SD2 | 0.611 | 0.820 | 0. [symbol] 01 | 0.841 | 0.879 | 0.817 |
| Total Power (AR) | 0.452 | 0.820 | 0.281 | 0.819 | 0.893 | 0.800 |

[symbol]

indicates data missing or illegible when filed

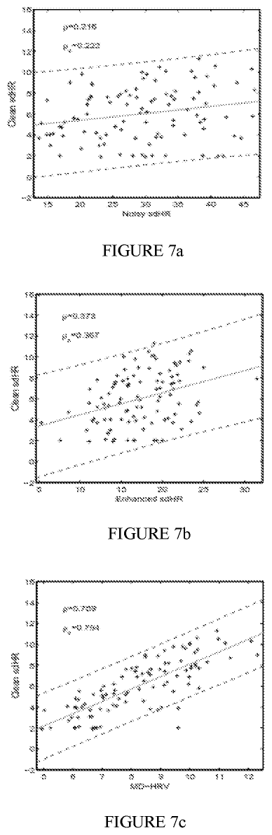

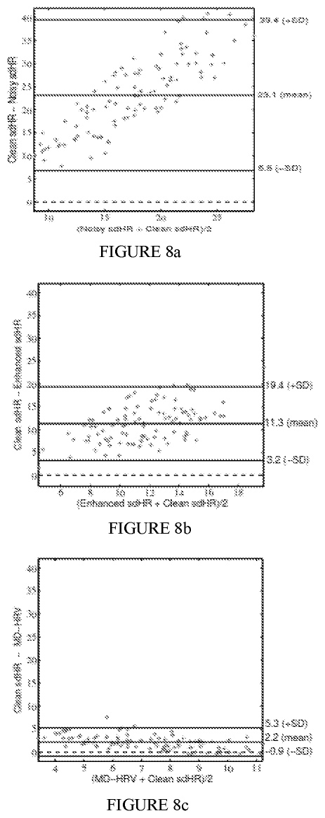

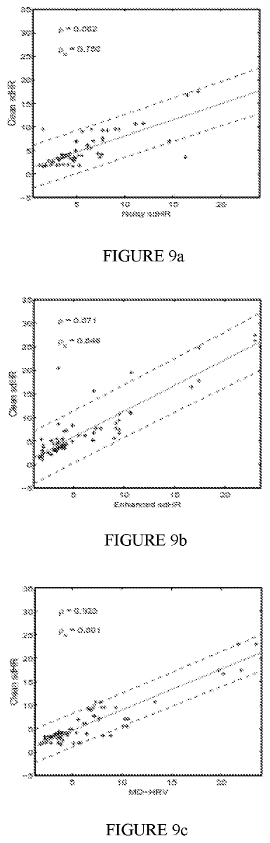

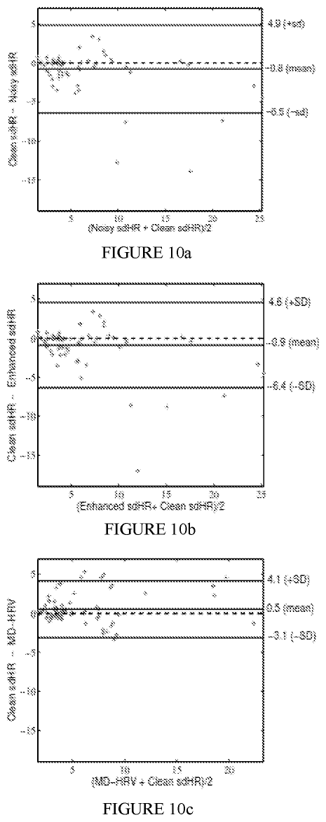

FIGS. 7 (a)-(c) further depict the scatterplots (and linear fit curves) of the sdHR metric computed from the noisy (subplot a) and enhanced signals (subplot b), as well as the MD-HRV metric computed from the noisy signals (subplot c). To avoid cluttered plots, only 100 noisy signals at 100 bpm and SNR=−10 dB are shown, thus representing scenarios similar to high levels of exercise and movement. Furthermore, FIGS. 8 (a)-(c) show the Bland-Altman plots for the three cases mentioned above, respectively. Lastly, FIGS. 9 and 10 show the scatterplots and Bland-Altman plots for Group 2 (80±10 bpm) in dataset 2 (recorded), respectively. As above, the sdHR benchmark metric computed from the noisy signal, the enhanced signal, as well as the proposed MD-HRV metric computed from the noisy signal are shown in the plots. As can be seen from the plots, the proposed MD-HRV metric results in higher correlations, tighter regression confidence intervals, and more compact Bland-Altman plots around zero (vertical axis) than the sdHR benchmark metric in both cases with and without wavelet enhancement.

### IV. Discussion

In the above description, a new noise-robust HRV metric was proposed based on a spectro-temporal ECG representation termed ‘modulation spectrum.’ The proposed metric was compared to six benchmark HRV indices computed using time-domain, frequency-domain and non-linear methods reported in the literature, with and without ECG enhancement.

From Table I, it can be seen that benchmark HRV metrics computed from noisy synthetic signals are generally well correlated with the true HRV values computed from the clean ECG signals for SNR levels greater than 0 dB. For extremely low SNR levels below −5 dB, however, typical of those observed with wearables during intense exercise, HRV measurement accuracy degrades quickly. Wavelet-based enhancement, in turn, was shown to be useful for most metrics, particularly for SNRs below −8 dB.

The proposed MD-HRV metric, on the other hand, computed only from the noisy ECG signal, showed stable correlation values generally greater than 0.9 up to an SNR=−8 dB, with a drop to around 0.71-0.76 at an SNR=−10 dB. In this extremely noisy scenario, it was difficult to accurately detect the main lobe in the modulation spectrogram (corresponding to the heart rate, see Section II-A), thus compromising HRV measurement. Notwithstanding, it can be seen that with the SDNN metric, while wavelet enhancement improved p by about 99% relative to the noisy case, the proposed metric outperformed the noisy case by 253%. Similar gains were seen across all of the benchmark metrics, including SDANN (177% compared to 63% post enhancement), sdHR (239% compared to 27% post enhancement), SD1 and SD2 (385% and 184% compared to 161% and 68% post enhancement, respectively), and total power (266% compared to 78% post enhancement). Overall, the correlation values obtained with the MD-HRV metric at an SNR=−10 dB are in line with those obtained with the benchmark metrics post enhancement at −8 dB≤SNR≤−5 dB. Closer inspection showed that the total power HRV benchmark metric resulted in the lowest correlation with MD-HRV across all tested SNR levels. This benchmark metric showed to be the most sensitive to artifacts, and resulted in the lowest correlation values of all benchmark metrics even for SNR=10 dB.

These results are further validated from the scatter and Bland-Altman plots shown in FIGS. 7 and 8, respectively, for the proposed and sdHR (pre and post enhancement) metrics; plots correspond to 100 randomly-selected ECG signals at 100 bpm and an SNR=−10 dB. The Bland-Altman plots, for example, show that the discrepancies between the true sdHR (computed from the clean signals) and the sdHR computed from the wavelet enhanced signals had a mean difference of 11.3±8.1, whereas this difference was of 2.2±3.1 for the proposed MD-HRV metric computed from the noisy signals. Overall, results from Table I and FIGS. 7 and 8 suggest that the proposed metric could be useful in extreme conditions where wearable ECG signals are highly contaminated with motion artifacts, such as during peak performance training.

Table II, in turn, reported results obtained from ambulatory recorded ECG data. Relative to Table I, the correlation values suggest a fair amount of noise is present in the recordings. Interestingly, wavelet based enhancement did not result in performance improvements for most of the benchmark metrics, thus highlighting the limitations of existing enhancement algorithms for data recorded in real-world settings. Notwithstanding, the proposed metric achieved reliable results without the need for a priori enhancement; overall, ρ and ρ_{S} values stayed between 0.8-0.9 for all six benchmark metrics. Overall, an average gain in ρ of approximately 49% was observed with the proposed MD-HRV metric over the wavelet-enhanced case. As in the case for synthetic ECG signals, the frequency-domain HR benchmark metric showed to be the most sensitive to artifacts. In fact, for Dataset 2 (recorded), wavelet enhancement deteriorated benchmark HRV measurement performance, likely due to the introduction of additional unwanted artifacts post enhancement. As with the synthetic signals, the scatter and Bland-Altman plots in FIGS. 9 and 10 show smaller confidence intervals and tighter distributions around the null bias, respectively. Overall, the proposed MD-HRV metric achieved a mean difference of 0.5±3.6 with the true sdHR computed from the original heart rate labels, thus comparing favorably to the −0.9±5.5 obtained with the wavelet enhanced benchmark metric. These findings suggest that the proposed metric is not only robust to noise, but also to different cardiac pathologies, thus is an ideal candidate for patient monitoring with ambulatory ECGs.

In one embodiment, the MD-HRV metric may be an improved noise-robust surrogate to existing HRV metrics with a small computational footprint. This can have important benefits for athletic and fitness applications in which SDNN, SDANN, SD1, SD2 and total power metrics have been widely used.

### V. Conclusion

In the above description, there is proposed a method of estimating HRV in extremely noisy settings. The proposed metric is based on the spectro-temporal representation of the ECG signal, commonly termed “modulation spectral” representation. The proposed metric, termed MD-HRV (modulation domain HRV), was extensively tested using both synthetic and recorded noisy ECG signals, and compared to six benchmark HRV metrics computed from the noisy, as well as enhanced ECG signals, which had artifacts removed using a state-of-the-art wavelet-based algorithm. MD-HRV, computed from the noisy signal, was shown to outperform benchmark metrics computed from the enhanced signals across different figures-of-merit, thus showing the potential of the metric for HRV measurement in extreme conditions with low-cost portable devices.

The embodiments of the invention described above are intended to be exemplary only. The scope of the invention is therefore intended to be limited solely by the scope of the appended claims.

1. A computer-implemented method for evaluating a heart rate variability comprising:
receiving an electrocardiogram representing an electrical activity of a heart;
determining a modulation spectrum for the electrocardiogram, the modulation spectrum comprising a plurality of per-frame modulation spectrograms each representing a frequency as a function of a modulation frequency;
for each per-frame modulation spectrogram, determining a respective central modulation frequency;
determining an indication of the heart rate variability using the determined respective central modulation frequencies for the per-frame modulation spectrograms, a mean central modulation frequency and the number of per-frame modulation spectrograms; and
outputting the indication of the heart rate variability.

2. The computer-implemented method of claim 1, wherein said determining the modulation spectrum comprises:
applying a first transform to the electrocardiogram, thereby obtaining a time frequency representation of the electrocardiogram; and
applying a second transform across a time dimension of the time frequency representation, thereby obtaining the modulation spectrum.

3. The computer-implemented method of claim 2, further comprising dividing the electrocardiogram into a plurality of overlapping time windows before applying the first transform and dividing the time frequency representation into a plurality of overlapping modulation windows before applying the second transform.

4. The computer-implemented method of claim 2, wherein the first transform is a first fast Fourier transform.

5. The computer-implemented method of claim 2, wherein the second transform is a second fast Fourier transform.

6. The computer-implemented method of claim 1, wherein said determining the respective central modulation frequency comprises determining a central modulation frequency of a first lobe contained in the per-frame modulation spectrogram.

7. The computer-implemented method of claim 6, further comprising identifying the first lobe for each per-frame modulation spectrogram.

8. The computer-implemented method of claim 1, further comprising calculating the mean central modulation frequency.

9. The computer-implemented method of claim 1, wherein said determining the indication of the heart rate variability comprises calculating a standard deviation of the central modulation frequencies of the per-frame modulation spectrograms.

10. The computer-implemented method of claim 1, further comprising measuring the electrocardiogram.

11. A system for evaluating a heart rate variability, comprising:
a spectrum calculator for receiving an electrocardiogram representing an electrical activity of a heart and determining a modulation spectrum for the electrocardiogram, the modulation spectrum comprising a plurality of per-frame modulation spectrograms each representing a frequency as a function of a modulation frequency;
a central frequency calculating unit for determining, for each per-frame modulation spectrogram, a respective central modulation frequency; and
a heart rate variability determining unit for determining an indication of the heart rate variability using the determined respective central modulation frequencies for the per-frame modulation spectrograms, a mean central modulation frequency and the number of per-frame modulation spectrograms and outputting the indication of the heart rate variability.

12. The system of claim 11, wherein the spectrum calculator is adapted to apply a first transform to the electrocardiogram, thereby obtaining a time frequency representation of the electrocardiogram and apply a second transform across a time dimension of the time frequency representation, thereby obtaining the modulation spectrum.

13. The system of claim 12, wherein the spectrum calculator is further adapted to divide the electrocardiogram into a plurality of overlapping time windows before applying the first transform and divide the time frequency representation into a plurality of overlapping modulation windows before applying the second transform.

14. The system of claim 12, wherein the first transform is a first fast Fourier transform.

15. The system of claim 12, wherein the second transform is a second fast Fourier transform.

16. The system of claim 11, wherein the central frequency calculating unit is adapted to determine a central modulation frequency of a first lobe contained in the per-frame modulation spectrogram.

17. The system of claim 16, wherein the central frequency calculating unit is further adapted to identify the first lobe for each per-frame modulation spectrogram.

18. The system of claim 11, wherein the heart rate variability determining unit is further adapted to calculate the mean central modulation frequency.

19. The system of claim 11, wherein the heart rate variability determining unit is adapted to calculate a standard deviation of the central modulation frequencies of the per-frame modulation spectrograms.

20. The system of claim 11, further comprising a sensor for measuring the electrocardiogram.
